(19) United States
(12) Patent Application Publication
(10) Pub. No.: US 2022/0249403 A1
(43) Pub. Date: Aug. 11, 2022

(54) METHOD FOR CARING FOR THE SKIN OR COAT OF ANIMALS

(71) Applicant: Ceva Santé Animale S.A., Libourne (FR)

(72) Inventors: Mélanie Rougier, Libourne (FR); Claudine Zemirline, Libourne (FR); Elodie Olivier, Libourne (FR)

(21) Appl. No.: 17/621,959

(22) PCT Filed: Jun. 26, 2020
(86) PCT No.: PCT/EP2020/067990

§ 371 Date: Dec. 22, 2021

(30) Foreign Application Priority Data
Jun. 26, 2019 (EP) 19305857.5

(51) Int. Cl. A61K 31/133; A61P 17/04; A61K 9/00

(52) U.S. Cl. CPC A61K 31/133; A61K 9/0017; A61P 17/04

(57) ABSTRACT

The invention concerns a method for caring for the skin or coat of animals, comprising the application of a rinse-off shampoo, followed by at least four consecutive applications of a composition in the form of a non-rinse-off foam. The invention also concerns a kit, in particular intended to implement a method for caring for the skin or coat of animals and, more specifically, to help reduce their itching.

### FIELD OF THE INVENTION

The invention relates to a method for caring for the skin or coat of pets, comprising the application of a composition of a rinse-off shampoo type, followed by at least four consecutive applications of a composition in the form of a non-rinse-off foam. The invention also relates to a kit, in particular intended to implement the method for caring for the skin and/or the coat of pets, and in particular for helping to reduce their itching, comprising said compositions in compartments.

### PRESENTATION OF THE PRIOR ART

Pets, such as dogs and cats, frequently have skin and coat problems, or cutaneous or coat issues, such as: dry skin, flaking, pruritus, itching, skin irritation, excessive loss of hair, and a dull coat. The problems of skin itching vary from very slight or occasional itching to extremely severe or almost continuous itching.

In animals, atopic dermatitis is a chronic inflammatory and itchy dermatosis having multiple origins. This disorder results from complex interactions between factors of genetic predisposition and environmental factors. The first signs of this disorder in dogs or cats generally appear between 6 months and 3 years. The damage mainly affects the face, the extremities, the deep folds, and the abdomen. The disorders of the atopic dermatitis or pruritus type associated with allergic dermatitis are usually treated by antipruritics, antihistamine agents, anti-inflammatories, or immunomodulators, such as ciclosporin, corticoids or oclacitinib. These treatments are generally accompanied by support or care treatments, such as treatments by shampoo, in order to achieve better, more rapid results, or in order to prevent or slow relapses.

However, in a general manner, it has been found that the treatments accompanying the care using shampoo are rarely followed by the animals' owners, because it is impractical to perform then since they require the shampoo to be rinsed, which can be particularly difficult when the animals have long hair or are of a nervous disposition. The repeated application of rinse-off shampoos can also reduce the effectiveness of topical treatments against tics and fleas, in particular if a time lag between the application of the anti-parasitic agent and the cutaneous care is not complied with.

There is thus a need to develop protocols for caring for the skin or the coat of pets, which are easy to implement while being as active, or indeed more active, than the protocols known hitherto.

The applicant has developed a particular protocol for applications of compositions for the skin or the coat of pets which is simple to implement, since it comprises just one application of a rinse-off shampoo during said protocol, said single application of shampoo being followed by applications of compositions which do not require rinsing. This protocol makes it possible to obtain very satisfactory results, in particular in terms of reducing itching, while making the treatment easier to use and more attractive for owners, thus improving the adherence to the treatment.

### SUMMARY OF THE INVENTION

The invention relates to a method for caring for the skin or coat of an animal, comprising the application, to the skin, of a rinse-off shampoo, followed by at least four consecutive applications, to the skin or the coat, of a composition in the form of a non-rinse-off foam.

In particular, the method comprises the application of a rinse-off shampoo, followed by 5, 6, 7 or 8 consecutive applications of a composition in the form of a non-rinse-off foam. In some embodiments, the shampoo and the composition in the form of a non-rinse-off foam are compositions comprising at least one compound that is intended for reducing the itching of animals, and in particular pets, such as dogs or cats.

The invention also relates to a kit comprising, in one or more compartment(s), at least one rinse-off shampoo and at least one composition in the form of a non-rinse-off foam, and optionally instructions setting out the method for using said shampoo and composition in the form of a non-rinse-off foam, as defined in the present invention.

### FIGURES

FIG. 1 shows the “control” protocol (A) and the protocol according to the invention (B) implemented in example 1

FIG. 2 shows the results of studies (A) and (B) of the example (average CADESI scores). A) Average CADESI score of the two studies. —CADESI-04 in % of change with respect to D0—average confidence interval (CI) at 95%). The horizontal lines represented by dashes (---) correspond to the values delimiting the degree of severity (in remission/average/moderate). The curve represented by broken lines ( . . . ) is that of group (A). The curve represented by a continuous line (_) is that of group (B). B) The CADESI scores of the two groups (A) and (B) of the studies of the example are presented in the form of percentage changes ((Dx-D0/D0)X100). The horizontal line represented by dashes (---) corresponds to a threshold score of 40%. The curve represented by broken lines (_) is that of group (A). The curve by _{[GH1][AH2]} a continuous line (_) is that of group (B).

### DETAILED DESCRIPTION OF THE INVENTION

The present invention thus relates to a method for caring for the skin or coat of animals, in particular pets, comprising the application of a rinse-off shampoo to the skin or the coat of an animal, followed by at least four consecutive applications, to the skin or the coat of said animal, of a composition in the form of a non-rinse-off foam.

The method applies to the skin or the coat of animals, according to the invention, can be implemented before, during or after a treatment of animals having cutaneous or coat issues, and in particular disorders of the atopic dermatitis or pruritus or seborrhoea type. The method of the invention can thus be an accompanying or support treatment for a therapeutic treatment, in particular treatment using antipruritic agents, antihistamine agents, anti-inflammatory agents, or immunomodulator agents, such as corticoids, ciclosporin or oclacitinib. Thus, the animals to be treated according to the method of the present invention may have been previously diagnosed as atopic. They may possibly have a period of remission of the clinical evidence of atopic dermatitis. They may be undergoing treatment for their atopic condition, in particular treated using antipruritic agents, antihistamine agents, anti-inflammatory agents, or immunomodulator agents.

The method according to the invention can be carried out on animals exhibiting itching or having exhibited itching, possibly independently of a therapeutic treatment, in particular systemically or in particular by injection or orally, in particular using antipruritic, antihistamine, anti-inflammatory, or immunomodulator agents. Animal itching may be very slight or sporadic, slight or somewhat more frequent, moderate or present at regular intervals, severe or present for prolonged periods, or extremely severe _{[GH3][AH4]}or present almost continuously.

According to a particular embodiment, the method of the invention makes it possible to reduce the itching of the animals (or also referred to as non-human mammals), in particular pets, such as dogs or cats.

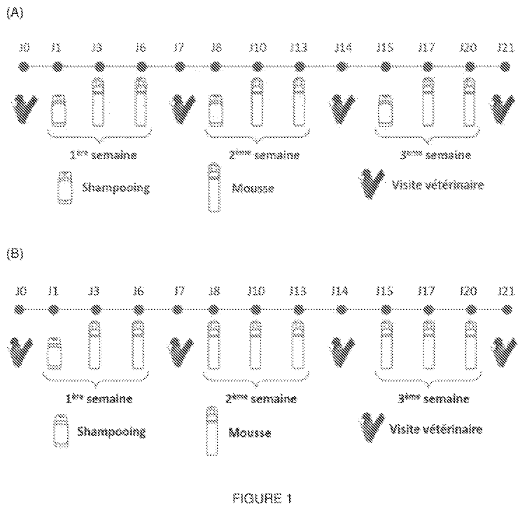

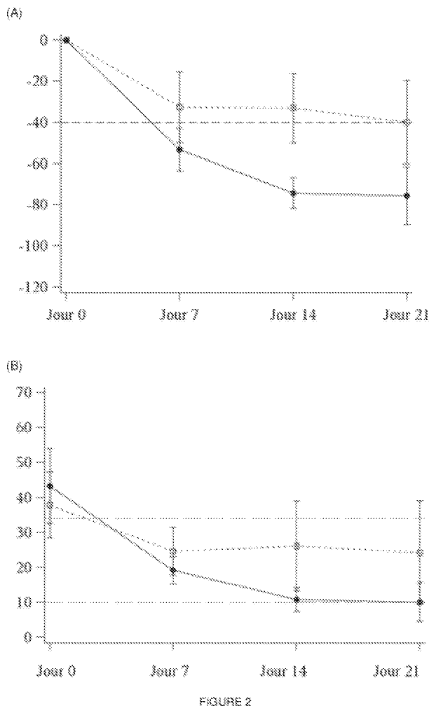

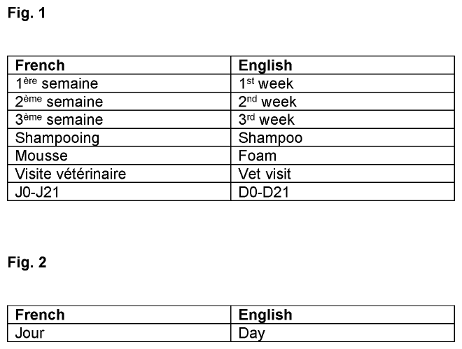

According to a particular embodiment, the method according to the invention is characterized in that each application (following the first application) is carried out at an interval of at least 24 hours, or preferably at least 48 hours, from the preceding application. The method is preferably carried out such that each of the consecutive applications of foam following the first application of rinse-off shampoo is carried out between 24 hours and 72 hours after the preceding application. Even more preferably, each of the consecutive applications of foam following the first application of rinse-off shampoo is carried out between 36 hours and 72 hours, and in particular between 48 hours and 72 hours, after the preceding application. The time intervals between each application of non-rinse-off foam can thus vary between 24 hours and 72 hours, in particular between 36 hours and 72 hours, and more particularly between 48 hours (2 days) and 72 hours (3 days). It can in particular be a protocol as described in FIG. 1.

Thus, more specifically, the method according to the invention comprises the following steps: application of shampoo (D1), application of the composition in the form of a non-rinse-off foam (D3) carried out 48 hours from the shampooing (D1), followed by an application of the composition in the form of non-rinse-off foam (D6) carried out 72 hours after D3, followed by an application of the composition in the form of a non-rinse-off foam (D8) carried out 48 hours after D6, followed by an application of the composition in the form of a non-rinse-off foam (D10) carried out 48 hours after D8, followed by an application of the composition in the form of a non-rinse-off foam (D13) carried out 72 hours after D10, followed by an application of the composition in the form of a non-rinse-off foam (D15) carried out 48 hours after D13, _{[GH5][AH6]}followed by an application of the composition in the form of a non-rinse-off foam (D17) carried out 48 hours after D15, followed by an application of the composition in the form of a non-rinse-off foam (D20) carried out 72 hours after D17.

According to a preferred embodiment, the method comprises the application of a rinse-off shampoo, followed by 4, 5, 6, 7 or preferably 8 consecutive applications of a composition in the form of a non-rinse-off foam. Thus, more specifically, the 4, 5, 6, 7 or preferably 8 consecutive applications of non-rinse-off foam according to the invention are not interrupted by an application of shampoo.

The method according to the invention may extend over a period of approximately 18 to 21 days. It can be repeated once, twice, _{[GH7][AH8]} or three times, or indeed more, if necessary. Thus, according to a particular embodiment, the method further comprises, after said at least 4, 5, 6, 7, or 8 consecutive applications of a composition in the form of a non-rinse-off foam, as defined above, at least one step consisting in an application of a rinse-off shampoo, it being possible for said shampoo application to be followed by consecutive applications of a composition in the form of a non-rinse-off foam, it being possible in particular for said shampoo application to be followed by at least 4 consecutive applications of a composition in the form of a non-rinse-off foam, as described in the present invention. According to a more particular embodiment, said additional step can be repeated several times, in particular once, twice, _{[GH9][AH2 ]} or three times.

As used here, the term “shampoo” includes any composition intended to clean the hair to the skin of non-human mammals, and requires rinsing with water after application to the animal. Generally, the shampoos have certain essential characteristics. The shampoos used in the present invention must of course be compositions that are well tolerated and are intended for topical application in the case of non-human mammals. A shampoo has moistening, foaming and detergent properties, in order to ensure satisfactory cleaning of the coat or of the skin. In a general manner, a shampoo composition of the invention contains surfactants, preferably at least one anionic, cationic, amphoteric, or polar non-ionic surfactant, or indeed a mixture thereof.

The anionic surfactants include in particular: the alkyl sulfates (such as the magnesium or sodium lauryl sulfates), the alkyl ether sulfates (such as lauryl ether sulfate), the alkyl polyether sulfates, the alkyl sulfonates (the alkyl groups having from 8 to 18 carbon atoms), fatty acid soaps, monosulfosuccinates of fatty alcohols, condensation products of fatty acids with methyltaurine, condensation products of fatty acids with sarcosine, and condensation products of fatty acids with a protein hydrolysate.

The cationic surfactants include in particular: the long-chain quaternary ammonium compounds, the esters of fatty acids and of amino acids, and the polyether amines.

The polar non-ionic surfactants include in particular: the esters of polyols and of sugars, the condensation products of ethylene oxide with fatty acids, with fatty alcohols, with long-chain alkylphenols, with long-chain mercaptans, and with long-chain amides, and the polyethers of polyhydroxylic fatty acids.

The amphoteric surfactants include in particular: the derivatives of amino acids, such as the derivatives of asparagine, the condensation products of monochloroacetic acid with imidazolines and alkylamine propionates, more specifically derivatives of betaine or imidazoline.

In the shampoo compositions, the surfactant composition is preferably between 5 and 50 by weight with respect to the total weight of the composition. The shampoos according to the invention can also contain other ingredients conventionally used in such compositions, such as viscosity-increasing agents, in particular acrylate/methacrylate copolymers, oils, waxes, vegetable extracts, emollients, carbomers, gums, in particular xanthan gum, polyethylene glycols, cyclomethicones, conditioners, preservatives, such as methyl parahydroxybenzoate or propyl parahydroxybenzoate, colorants, perfumes, or mixtures thereof.

The non-rinse-off foams are thus suitable for a non-rinsed application, i.e. the compositions are not removed by rinsing, and in particular by rinsing with water, after being applied. The non-rinse-off foams generally comprise a low surfactant composition, generally from 1 to 10%, and preferably from 2 to 4% by weight, with respect to the total weight of the composition.

The non-rinse-off foams used in the present invention must of course be compositions that are well tolerated and are intended for topical application in the case of non-human mammals.

The non-rinse-off foams that can be used in the present invention may be of different types.

They may in particular be:
Aerosols, in which the foam is generated by a propellent gas, foams referred to as an aerosol or spray,
Overrun creams, in which air bubbles are introduced into the product via a particular manufacturing method,
Foaming formulas low in foaming surfactants but packaged in a packaging provided with a mechanical foam generator system (pump with a grate of the Pulvorex type).

The non-rinse-off foams are preferably foams referred to as an aerosol or spray.

As used here, the “non-human mammals” or “animals” include all mammals with the exception of human beings. The non-human mammals are preferably pets, including, in a non-limiting manner, domestic animals such as in particular dogs, cats, rabbits, ferrets, and hamsters. In a more preferred embodiment of the invention, the non-human mammals are cats, and preferably dogs.

The shampoo compositions and/or non-rinse-off foam compositions used according to the invention are intended for cleaning or washing the skin and/or the coat. They can be used for maintaining the health of the skin or the coat of animals, in particular by maintaining the cutaneous balance, by maintaining or improving the hydration of the skin, by soothing the skin, or indeed by reducing or soothing sensations of itching or irritation. Thus, in particular, the method for caring for the skin or the coat of animals, according to the invention, is a method for cleaning or washing the skin and/or the coat, for maintaining the health of the skin or of the coat of the animals, for maintaining the cutaneous balance, for maintaining or improving the hydration of the skin, for soothing the skin, or indeed for reducing or soothing sensations of itching or irritation.

According to a preferred embodiment of the present invention, the shampoo compositions and/or non-rinse-off foam compositions used according to the invention further comprise at least one compound which is intended for treating or caring for the skin or the coat of animals. It may in particular be at least one compound having hydrating or restructuring properties, a compound intended for improving the barrier effect of the skin or restoring the barrier effect thereof, or intended for eliminating or reducing dandruff or excess sebum, or indeed intended for reducing itching or helping to reduce dermatitis in pets. The shampoo compositions and/or non-rinse-off foam compositions used according to the invention may comprise, as active compounds, antimicrobials (compound limiting the spread of bacteria, yeasts or fungi), or disinfectants, such as chlorhexidine.

The compounds intended for treating or helping to care for the skin or the coat of animals may also be for example compounds of the type having a sphingoid base, or indeed plant extract compounds.

In particular, the active compounds present in the compositions of the method according to the invention, i.e. the shampoo and the non-rinse-off foam compositions, are identical or different; preferably, they are identical.

In the shampoo composition and/or non-rinse-off foam composition of the invention, the composition of active compound intended for treating or helping to care for the skin or the coat is preferably between 0.001 and 30% by weight with respect to the total weight of the composition. The most preferred concentrations may vary from 0.005 to 20% by weight, 0.01 to 10% by weight, 0.02-5% by weight, or 0.05 to 3% by weight.

Compounds obtained from a plant which are capable of acting on different factors of animal atopic dermatitis are particularly suitable for helping to reduce itching of animals. It is in particular a compound obtained from Ophiopogon japonicus, such as described in particular in the patent application WO2017121965. Said compound is particularly suitable for use, by topical application to the skin or coat of animals, in a treatment accompanying the care of atopic animals. It can be used on atopic skins, in particular for helping to reduce or slow the relapse rate, to reduce the severity score of atopic dermatitis in animals (CADESI score), to reduce the intensity and/or the frequency of itching or of pruritus, and more generally for improving the quality of life of the animal and/or its owner.

Within the meaning of the invention, “active compound obtained from Ophiopogon japonicus” means any molecule or mixture of molecules obtained from Ophiopogon japonicus. They may be native molecules of the plant, or molecules obtained by any kind of transformation of native molecules of the plant, for example by hydrolysis. The active compound according to the invention is preferably a hydrolysate.

“Hydrolysate” means any extract originating from Ophiopogon japonicus which is obtained by means of a method comprising at least one step of enzymatic or chemical hydrolysis of

Ophiopogon japonicus, preferably at least one step of enzymatic hydrolysis. “Ophiopogon japonicus” means all or part of the plant. It may be the entire plant, or a part of the plant. It is preferably tubers of Ophiopogon japonicus.

Ophiopogon japonicus is a perennial, low and abundant herbaceous species having rhizomes, from the family of the Liliaceae.

The sphingoid base is preferably selected from the group consisting of sphingosine, sphinganine and phytosphingosine. Phytosphingosine is most particularly preferred.

Suitable examples of derivatives of sphingoid bases include N-lactyloyl-phytosphingosine, _{[GH11][AH12]}N-salicyloyl-phytosphingosine or N-retinoylphytosphingosine, i.e. compounds which are N-substituted.

Other suitable examples of derivatives of sphingoid bases include the salts of sphingoid bases. The anion of the salt may be derived from any suitable acid, i.e. from those acids which, after being mixed with the sphingoid base in a suitable solid, produce a salt having improved solubility in water. The acids which are themselves effective when they are applied to the coat or the skin are preferred. The preferred salts of the sphingoid base, to be used in the composition of the present invention, are the salts which can be obtained using acids which, when mixed with the above-mentioned sphingoid base in a suitable solvent, produce a salt having an increased hydrosolubility with respect to the solubility in water of the sphingoid base per se. The salts of phytosphingosine are preferred for improved solubility and improved bioavailability.

In a preferred embodiment, the acid is a hydrophilic acid which is capable of delivering the sphingoid base salt to the aqueous phase of the veterinary composition. The suitable hydrophilic acids include a-hydroxy alkanoic acid, a 3-hydroxyalkanoic acid, an α,p-dihydroxy alkanoic acid, an alkanedioic acid, or a mineral acid. Examples of preferred hydrophilic organic acids are lactic acid, glycolic acid, malic acid, pyruvic acid, succinic acid, fumaric acid, ascorbic acid, gluconic acid and/or pyroglutamic acid; examples of preferred mineral acids are hydrochloric acid, nitric acid, and/or phosphoric acid.

In another preferred embodiment, the acid is a lithophilic organic acid for making it possible to increase the effectiveness of the lithophilic acid and of the sphingoid base in the sphingoid base salt.

It goes without saying that the method according to the invention may comprise one or more steps in addition to those cited above, for example preceding or following steps. A repetition of the successive steps of the method according to the invention can also be envisaged.

Thus, the protocol set out in FIG. 1(B) can be repeated as many times as necessary, without the need for a period of interruption, if considered appropriate.

The invention additionally relates to a kit comprising, in one or more compartment(s), at least one rinse-off shampoo and at least one composition in the form of a non-rinse-off foam, and optionally instructions setting out the method for using said shampoo and composition, as defined in the present invention. The composition in the form of a rinse-off foam may be present in just one compartment or in a plurality of compartments. In the latter case, each compartment preferably comprises a composition in the form of a non-rinse-off foam corresponding to each of the at least four consecutive applications. Thus, the kit comprises at least four compartments, each comprising a composition in the form of a non-rinse-off foam, and another compartment comprising the rinse-off shampoo.

The invention will be better understood from the following examples, which are illustrative and non-limiting.

### Example 1:Treatment of Dogs According to the Present Invention Compared with a Conventional Protocol

The aim of this study was to evaluate the effectiveness of an association of shampoo and foam comprising phytosphingosine, from the range of products (Douxo Calm®, sold by the company Ceva Santé Animale) during a relapse of atopic dermatitis in dogs. In order to carry out these tests, for each study 6 to 9 dogs, which had been previously diagnosed as atopic, receiving concomitant treatment (oclacitinib or ciclosporin) and exhibiting a clinical relapse of atopic dermatitis, were treated for 3 weeks.

The treatment consisted, for study (A) (control), in applications of a shampoo on D1, D8 and D15, and in twice weekly applications of a non-rinse-off foam between the shampoo applications on D1, D8 and D15 (FIG. 1 (A)).

For study (B) (according to the invention), the treatment consisted in one single application of shampoo on D1, followed by applications every two-three days (48-72 hours) of a foam, for 21 days.

In order to assess the effectiveness of these studies, the CADESI-04 severity scores (Vet Dermatol. 2014, Apr.; 25(2):77-85, e25. doi: 10.1111/vde.12107. Epub 2014 Jan. 25) and the visual analog scales of pruritus were analyzed and recorded each week. The values obtained at the given times were compared with the starting situation on DO (1 day before the application of the first shampoo (D1)).

After 21 days of treatment, in study (A) 3/5 of the dogs (60%) treated exhibited a 40% reduction in the CADESI score, while 6/7 (85%) of the dogs of study (B) exhibited this same reduction.

These studies also show the effectiveness of the Douxo Calm® shampoos and foam used in the two studies, in parallel with a long-term immunomodulator treatment that was unchanged during the study. For the two groups (A) and (B), the CADESI scores exhibited a significant reduction after 21 days of treatment, with a more significant improvement for group (B) than for group (A).

FIG. 2A shows the average changes in the CADESI score for each of studies (A) and (B). In the two studies, a reduction in the CADESI score was observed during the treatment (FIG. 2A). After 21 days of treatment, the average percentage reduction was close to the provided threshold of −40% for group (A), and less than the threshold of −40% for group (B), which means that, in general, after 21 days of treatment the CADESI reduced on average by less than 40% (FIG. 2B). On day 21, the average percentages of CADESI variation are −39.8±23.1% for group (A), and −75.6±19.0% for group (B).

The main criterion was based on the percentage CADESI reduction between D0 and D21. Thus, the highest percentage obtained can be used as the main criterion distinguishing the groups tested. Different percentages were tested (see Table 1). Table 1 provides information on the number of dogs that had reached the specific percentage CADESI reduction. The threshold of 50% corresponds to the difference level observed between the two protocols; indeed, just one dog reached this threshold for group (A), while 6 out of 7 dogs reached it in group (B).

TABLE 1
Percentages of dogs reaching different percentage CADESI reductions

| | GROUP (A) | GROUP (B) |
|---|---|---|
| CADESI-04 reduction > 30% on D21 | 4/5 (80%) | 7/7 (100%) |
| compared with D0 | | |
| CADESI-04 reduction > 40% on D21 | 3/5 (60%) | 6/7 (85%) |
| compared with D0 | | |
| CADESI-04 reduction > 50% on D21 | 1/5 (20%) | 6/7 (85%) |
| compared with D0 | | |
| CADESI-04 reduction > 70% on D21 | 1/5 (20%) | 5/7 (71%) |
| compared with D0 | | |

1. A method for caring for the skin or coat of animals, comprising the application, to the skin, of a rinse-off shampoo, followed by at least four consecutive applications, to the skin or the coat, of a composition in the form of a non-rinse-off foam.

2. The method according to claim 1, characterized in that each application of a composition in the form of a non-rinse-off foam is carried out at an interval of at least 24 hours, or preferably at least 48 hours, from the preceding application.

3. The method according to either claim 1, characterized in that each of the consecutive applications of foam following the first application of rinse-off shampoo is carried out between 24 hours and 72 hours, preferably between 48 hours and 72 hours, after the preceding application.

4. The method according to claim 1, characterized in that the method comprises the application of a rinse-off shampoo, followed by 4, 5, 6, 7 or 8 consecutive applications of a composition in the form of a non-rinse-off foam.

5. The method according to claim 1, characterized in that the method comprises the application of a rinse-off shampoo, followed by 8 consecutive applications of a composition in the form of a non-rinse-off foam.

6. The method according to claim 1, characterized in that the method further comprises, after said at least 4 consecutive applications of a composition in the form of a non-rinse-off foam, at least one step consisting in an application of a rinse-off shampoo, it being possible for said shampoo application to be followed by consecutive applications of a composition in the form of a non-rinse-off foam, it being possible in particular for said shampoo application to be followed by at least 4 consecutive applications of a composition in the form of a non-rinse-off foam.

7. The method according to claim 1, characterized in that the shampoo and the composition in the form of a non-rinse-off foam are compositions comprising at least one compound that is intended for treating or helping to care for the skin or the coat of animals, in particular to reduce the itching of animals, in particular pets.

8. The method according to claim 1, characterized in that the method is a method for cleaning or washing the skin and/or the coat, for maintaining the health of the skin or of the coat of the animals, for maintaining the cutaneous balance, for maintaining or improving the hydration of the skin, for soothing the skin, or indeed for reducing or soothing sensations of itching or irritation.

9. A kit comprising, in one or more compartment(s), at least one rinse-off shampoo and at least one composition in the form of a non-rinse-off foam, and instructions setting out the method defined according to claim 1.
